(19) United States
(12) Patent Application Publication
(10) Pub. No.: US 2005/0171456 A1
(43) Pub. Date: Aug. 4, 2005

(54) Foot pressure and shear data visualization system

(76) Inventors: Gordon B. Hirschman, Cohoes, NY (US); Brian L. Davis, Moreland Hills, OH (US)

Correspondence Address: HESLIN ROTHENBERG FARLEY & MESITI PC, 5 COLUMBIA CIRCLE, ALBANY, NY, 12203, US

(21) Appl. No.: 10/767,761

(22) Filed: Jan. 29, 2004

(51) Int. Cl. A61B 5/103; A61B 5/117

(52) U.S. Cl. 600/592

(57) ABSTRACT

A foot pressure and shear visualization system employs a deformed mesh technique to visualize horizontal shear and color mapping to visualize vertical pressure on a skin surface, e.g. the plantar surface of a foot. Additional cues, such as scaled, directed arrows may be used to enhance the visualization. This system includes a set of sensor hardware interface modules to acquire foot visualization data sets, a data base manager that allows host applications to store and retrieve the data files in a data base, a graphical user interface that performs the display and control aspect, as well as maintains the data files, and a visualization and analysis engine that performs all data set rendering and analysis. The graphical user interface controls a main user interface window, while the visualization and analysis engine controls a rendering window for the combined shear and pressure visualization.

### BACKGROUND OF THE INVENTION

A. Field of the Invention

The present invention relates to methods, systems and computer products for providing a visualization of forces acting on the skin, and, more particularly, for presenting an accurate representation of three-dimensional pressure and shear patterns on the plantar surface of a foot and identifying and indicating areas of concern for pathological consequences to the skin.

B. Description of the Background Art

According to the American Diabetes Association, there are approximately 16 million patients in the United States with diabetes, with 800,000 new diagnoses each year. For these patients, foot complications account for more hospitalizations than any other clinical problem, and plantar ulcerations are a major reason for subsequent foot amputation.

In the etiology of foot ulcers in diabetic patients, it is generally agreed that non-mechanical factors such as peripheral neuropathy, dry skin, and/or vascular problems are often major contributing causes for skin ulceration, with neuropathy of prime importance. However, current opinion is divided as to the principal mechanical factors leading to ulcer formation; repetitive loads, shear and pressure all are cited as potentially detrimental. In a number of studies, the conclusions were that elevated plantar pressures constitute a major biomechanical factor in the etiology of skin ulceration. Another study showed that diabetic neuropathic ulceration occurred at sites of maximal shear stress under the foot. Unfortunately, the studies that are focused on the nature of shear forces under the feet have been limited by instrumentation that could detect shear forces in only one direction, (e.g., medio-lateral). This limitation inevitably results in an underestimation of maximum shear stress, since this quantity is a vector-addition of medio-lateral and anterior-posterior components.

Recent research indicates that proper clinical prophylactic and therapeutic measures will require understanding of the interaction of shear and pressure, since this is likely to be a key factor in the etiology of diabetic ulcers.

A number of companies currently offer devices to clinicians for evaluating plantar pressure patterns. Although shear injury can occur when there is relative horizontal displacement between the skin and underlying fatty tissues, the ability to measure shear has lagged behind the development of pressure platforms. From a biomechanical viewpoint, skin stresses are certainly dictated by both pressure and shear, although the combined effect is likely to be more complicated that a simple vector addition of these stresses would suggest. For instance, the likelihood of a skin site breaking down may not only depend on pressure and shear at that site, but on the nature of skin stresses at adjacent sites. Stress couples that tend to cause skin torsion may contribute to ulcer risk in a manner different to those stresses that act toward each other (“bunching”) or away from each other (“stretching”).

New hardware for quantifying both shear and pressure either already exists at major research institutes, or is the subject of R & D efforts in companies that market pressure platforms. With the increasing application of microelectromechanical systems, hardware systems for shear measurement are likely to reach commercial status in the near future. Companies are currently working on solving hardware issues that relate to measuring 3-D stresses at multiple sites under the foot. However, a need exists for advanced software packages that can display and analyze the large data sets likely to result from these devices. Only then can this new hardware be successfully marketed to potential users.

Incorporation of shear measurement capability into commercial measurement systems, which typically consists of a matrix of more than 2,000 pressure sensors, will further increase their complexity. Because of the nature and diversity of the sensors involved, as well as the volume of the data, clinical utility of these measurement systems depends upon the software for data analysis and display. Although the existing software works well for display and analysis of one-dimensional pressure data, new techniques are needed as measurement systems add the ability to measure shear stresses, creating a 3-D data set, albeit acting in a single plane. This critical issue must be addressed before shear and pressure hardware can be used in a clinical setting. Existing software to display and quantify pressure does not meet the needs of researchers and clinicians who want to understand the role of both shear and pressure.

### SUMMARY OF THE INVENTION

The present invention provides a method for visualizing force acting on a skin surface where the force is sensed at distributed points on the skin surface by an array of sensors. The visualization method comprises displaying an image of a mesh, and deforming the mesh in a plane of the mesh in accordance with the force sensed by the array of sensors. The image of the mesh may be superimposed on an outline of the skin surface. A second force acting on distributed points on said skin surface may also be sensed, and color mapping may be used to display the sensed second force along with said image.

In accordance with one aspect of the present invention, the sensed force may be shear, the second force may comprise force or pressure acting generally normal to the skin surface and the second force may be displayed by color mapping of the mesh or of a plane parallel to the mesh.

In another aspect, the present invention provides a method of visualizing forces acting on a skin surface, as detected at distributed points on said skin surface with an array of skin sensors. The method includes automatically providing a first representation of detected forces acting in a plane of said skin surface, and simultaneously automatically providing a second representation of detected forces acting in a direction generally normal to the skin surface. The first representation may comprise a mesh deformed within a plane of the mesh, and the second representation may comprise color mapping. The first representation may also include directed scaled arrows. Alternatively, the first representation and second representation may comprise common arrows having a vector direction and magnitude corresponding to a composite vector of all detected forces. The first representation may portray shear and the second representation may portray pressure. The method may further include a step of automatically determining a location of maximum value of at least one of: the detected forces, skin bunching, skin stretching and skin twisting, and highlighting of said location.

In a further aspect, a system for visualizing forces acting on a skin surface is provided. The system includes a visualization and analysis engine for receiving data files containing force readings from an array of sensors at distributed locations of said skin surface, analyzing said data files and generating a first representation of forces acting in a plane of said skin surface and a second representation of forces acting normal to said skin surface, plus a rendering window for simultaneously displaying the first and second representations. The system may also include a graphical user interface for user control of the visualization and analysis engine, a data base manager for managing storage, categorization and retrieval of data files, and sensor hardware interface modules for acquiring data files from a variety of different sensor hardware systems.

In another aspect, an article of manufacture comprising a computer usable medium having a computer readable program code means embodied therein for a system for visualizing forces acting on a skin surface, is provided. The article includes computer readable program means for establishing a visualization and analysis engine and computer readable program means for causing the visualization and analysis engine to simultaneously display first and second representations of detected forces in a rendering window.

In a still further aspect, the invention contemplates the use of the above described methods, system and computer product in combination with an array of sensors for detecting forces acting on a skin surface.

According to a preferred embodiment of the present invention, the above described needs are met and additional advantages provided, by a system, method and computer product which allow a clinician to see an accurate simultaneous representation of three-dimensional (3-D) pressure and shear patterns on an image of the plantar surface and identifies areas of concern. Adaptable to a variety of measurement systems, the present invention uses a deformed mesh technique to visualize horizontal shear and color mapping to visualize vertical pressure. Additional cues, such as scaled, directed arrows, are available as an option to enhance the visualization.

In a preferred embodiment, a rectangular grid is displayed representing the plane where the foot meets a sensor array. This grid is deformed at each line intersection according to the values of horizontal shear at the corresponding sensor location (or values interpreted from neighboring sensors). Vertical pressure is added by color mapping the grid or, preferably, a flat plane below the grid so that a range of pressures appear in a corresponding range of colors. This technique may also be enhanced by adding two dimensional (2-D) arrows on the plane of the grid. The arrows are oriented in the vector direction of the shear at a point, and the length of the arrow is proportional to the magnitude of the vector shear.

Alternatively, three-dimensional (3-D) arrows can be used to represent both shear and pressure. In this case, the arrow points to a 3-D coordinate corresponding to the x, y, z position calculated by scaling the horizontal shear (x, y) and the vertical pressure (z). The length of the arrow is proportional to the magnitude of the vector formed by the x, y shear and z pressure.

Yet another approach combines the 2-D shear indicating arrows above, with a color mapped grid that is elevated at each grid intersection proportional to the pressure at that x, y location. This combines an elevated grid technique for viewing pressure with the novel use of scaled, directed arrows in the horizontal plane for indicating shear.

The present invention also contemplates analysis, identification and visualization of areas of maximum shear and pressure, and patterns of shear representing bunching, stretching and twisting of skin on the plantar surface that may create a high risk of ulceration. The present invention computes bunching, stretching and twisting indices and identifies the locations where these indices are at a maximum both at a single instant in time and over an entire data set. In this way, sensor data can be automatically scanned and regions and times of interest highlighted for further inspection. Availability of the present invention, in combination with new pressure/shear hardware measurement systems, will greatly improve the diagnosis, prevention and treatment of foot ulcers in diabetic patients.

In terms of broader commercial potential, the present invention is applicable to situations where a need exists to evaluate the interface between skin and support surfaces more effectively. For example, the present invention can be extended to the estimated one million patients per year who develop pressure ulcer bedsores, as well as to prosthetic limb patients who are at risk of skin breakdown due to peripheral vascular disease.

The system architecture of the present invention will handle pressure and shear data from a variety of sensor systems and convert these to a unified internal data representation to support analysis and visualization. This unified data model will accommodate variations in spatial and temporal frequency between sensor systems. Since the key capabilities are imbedded in a central software engine, the architecture can be implemented either as a turn-key system or as an add-on for integration with software systems from other vendors. This modular design facilitates commercialization.

### BRIEF DESCRIPTION OF THE DRAWINGS

These and other features, aspects and advantages of the present invention are explained in greater detail hereinafter with reference to a preferred embodiment illustrated in the accompanying drawings, in which:

FIG. 1 illustrates a control panel and rendering window in accordance with the present invention;

FIG. 2 illustrates the effect of grid interpolation and scale factor on the visual presentation of shear;

FIG. 3 illustrates the use of directional arrows to enhance visualization of shear;

FIG. 4 depicts 3-D arrows showing shear and pressure;

FIG. 5 illustrates the use of grid deformation to visualize both shear and pressure;

FIG. 6 illustrates options for viewing pressure;

FIG. 7 illustrates an analysis numerical read-out window;

FIG. 8 is an example of maximum shear and pressure visualization;

FIG. 9 illustrates the foot pressure and shear data visualization system software architecture; and

FIG. 10 depicts a visualization and analysis engine architecture.

### DETAILED DESCRIPTION

The present invention provides a system, method and computer product for analyzing and visualizing distributed shear and pressure data, and, is especially useful, for generating and displaying an accurate simultaneous representation of 3-D pressure and shear patterns on the plantar surface of a foot, and for identifying areas of concern.

Although the system is compatible with a wide variety of shear and pressure measurement hardware, a prototype was initially designed to work with data generated by an experimental sensor array.

This sensor array (not shown) consists of a rectangular array of 8×10 sensors, each of which is 1.0×1.0 cm. The overall array size is suitable for measurement under the critical forefoot region. Each of the 80 sensors contains a set of strain gages that are sensitive to vertical pressure, x-direction shear, and y-direction shear. A personal computer (PC) handles data acquisition and sequentially scans the sensors, returning to each sensor 30 times per second. The strain gage voltage measurements are converted to vertical pressure, medio-lateral (M-L) shear, and anterior-posterior (A-P) shear by algebraic manipulation and application of a set of calibration data.

Initially, we used previously collected sample data from anonymous subjects that were preprocessed to produce calibrated pressure, M-L shear and A-P shear values in a comma-separated values (CSV) text file format that could be easily read by the prototype software. Although data from each sensor carries a unique timestamp because of the sequential scanning of the data acquisition computer, we grouped each complete scan of the entire 80-sensor array into a single frame of data for visualization and analysis. A complete experimental data set would typically include 30 to 50 such frames. To augment the available sensor data, we also generated synthetic data sets in the same format. The synthetic data allowed us to exaggerate features of interest for testing and evaluation.

The software was originally developed using the Visualization Toolkit (VTK) library with bindings to the tool command language (TLC). VTK is an open source, freely available, object-oriented software system for 3-D computer graphics, image processing, and visualization. Originally developed by researchers at GE's Corporate Research and Development Center, VTK is now supported by Kitware, Inc. (www.kitware.com), founded by two of the three original GE authors. TCL provides the simplest development environment for VTK applications, but can be awkward and difficult to maintain as an application grows in complexity. We chose to move development of the prototype software from TCL to C++ as we evolved from purely 2-D visualization of horizontal shear to a representation that combines horizontal shear and vertical pressure. C++ is the native language of the VTK package and affords the most control and the fastest program execution. It also was a more suitable language for development of mathematical analysis code than TCL.

To make this program control user friendly, we developed a Microsoft (MS) Windows-compatible graphical user interface (GUI) control window using MS-Visual Basic. Selection of Visual Basic allowed rapid development of a professional-looking user interface. The C++ code was packaged as a dynamic link library (DLL) that was called from the Visual Basic main program.

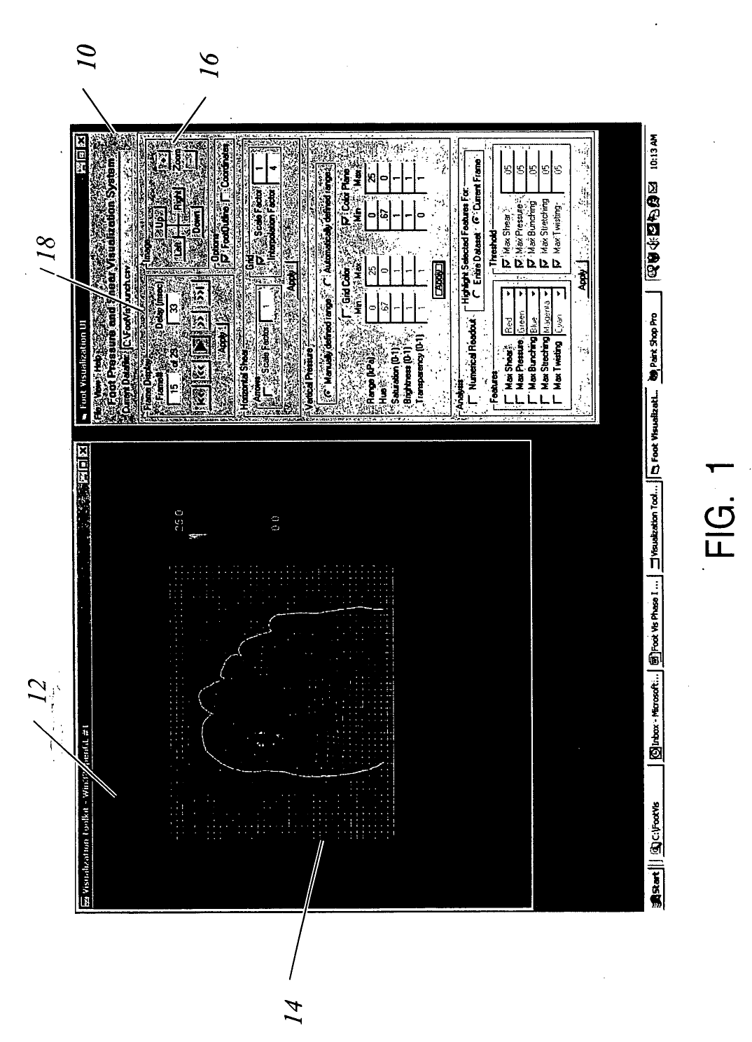

When invoked, the program displays two windows (see FIG. 1) that aid in visualization and analysis of sensor data sets: 1) a control panel window 10 produced by the Visual Basic main program, and 2) a rendering window 12 (produced by the C++ code using the VTK library) that contains a visual image 14 of the data. Both windows can be repositioned on the screen to facilitate viewing. The VTK library supports rotation and zoom of the image in the rendering window using the mouse buttons. From the control panel 10, the user can select and open a CSV data file and display the image 14 of the first frame in the rendering window 12. The image displayed can be shifted up, down, left, right, and can also be zoomed in and out using buttons 16 on the control panel. The control panel contains some frame display functions 18 that give the user the ability to easily view any frame of data in that data file, and to play the frames in an animated sequence of images. The control panel also allows the user to select several options to modify the visualization or invoke analysis using the techniques and algorithms described below. Algorithms and software to visualize shear and pressure data were developed as outlined in the following paragraphs.

Shear Data Visualization. A fundamental aim is to display horizontal shear data in conjunction with vertical pressure data. A deformed mesh 20 is used to represent horizontal shear. The mesh begins with a rectangular grid with the points at the intersection of lines corresponding to each sensor location. An additional pair of lines 21, 23 in each direction serves to frame the grid representing the sensor array and provide a fixed reference. The undeformed location of each intersection corresponds to the sensor location in the array. To visualize shear, the grid is deformed so that the x-y location of each line intersection is offset as follows:
X=xₒ+Aσₓ (1)
Y=yₒ+Aσ_{y} (2)
where
xₒ, yₒ is the location of the sensor σₓ is the stress in the x-direction
σ_{y} is the stress in the y-direction A=scale factor chosen for optimal visualization.
   Using data files representing a sensor array with 10 sensors in the M-L direction and 8 sensors in the A-P direction, the nominal visualization grid is comprised of a 12×10 matrix of points. The inner 10×8 matrix of points (places where the grid lines intersect) represent sensor locations, while the points on the outside edges represent anchor points used to create a reference that frames the image. Anchor points do not correlate to any sensors, and are represented with 0 shear and 0 pressure. The deformed grid is a very effective way of visualizing changes in shear, but is not very effective in seeing shears that are uniform across the foot. The frame lines connecting the anchor points help to interpret cases with high uniform shear since they are never moved from their original locations.

To enhance perception of the deformed mesh, the software provides for an interpolation factor that increases the resolution of the grid by using linear interpolation. For an M×N sensor array plus anchor points with an interpolation factor of F, the visualization grid is (F*(M+1))+1 by (F*(N+1))+1. Thus, for our 10×8 sensor array, when the interpolation factor is set to 1, a 12×10 grid matrix is created. An interpolation factor of 2 creates a 23×19 grid matrix, and an interpolation factor of 4 creates a 45×37 grid.

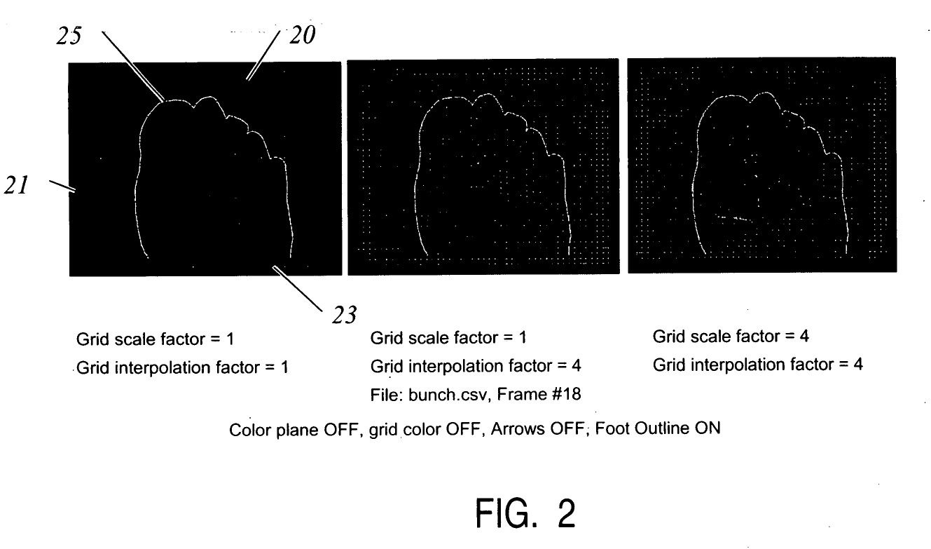

By changing the grid scale factor (the value of A in Equations 1 and 2), the grid deformation can be controlled. A value of zero completely disables grid deformation. Refer to the examples in FIG. 2, which illustrate how the interpolation factor and grid scale factor can affect an image. Note that the visualizations include a foot outline 25. This foot outline, which can be enabled (made visible) or disabled through the control panel, allows the user to gain a spatial perspective as to where the foot lies with respect to the shears and pressures that are being displayed. Initially, we overlaid a scanned image of a tracing of the foot outline on the stress and pressure data. However, where sensor resolutions are higher, software may be used to automatically extract a foot outline from the data.

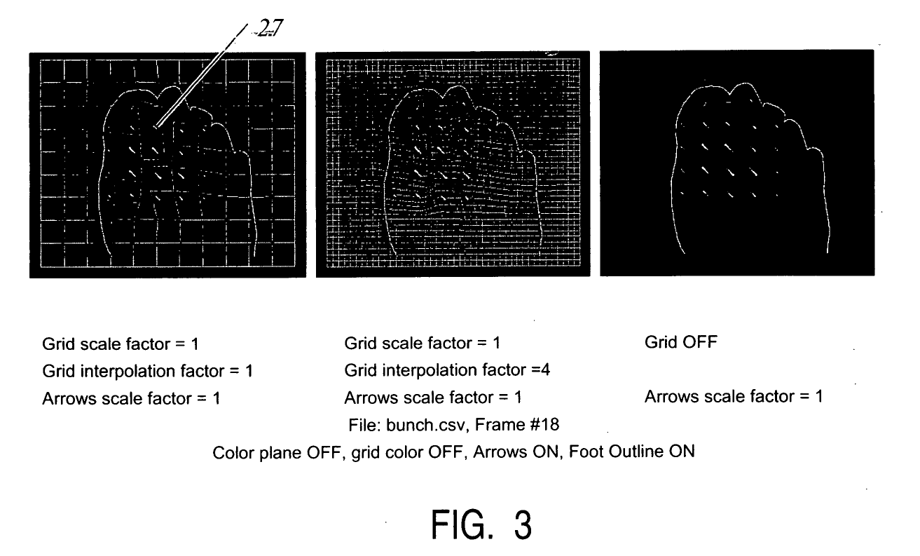

As an alternative to the deformed grid approach, we examined the use of a field of scaled arrows oriented in the direction of shear at each sensor location. Initially, we did not apply interpolation to the arrows, but rather maintained the correspondence to sensor locations. Alternatively, where sensor densities might vary considerably, we could interpolate or resample data to provide more or fewer arrows than sensors. The center of an arrow represents the sensor location, and it points in the direction of the shear vector. The relative magnitude of an arrow is proportional to the shear magnitude. The software provides a user-selectable arrow scale factor to change the length of all the arrows in the image. FIG. 3 provides examples of the arrows 27; note that several are so small that they are not visible in the images.

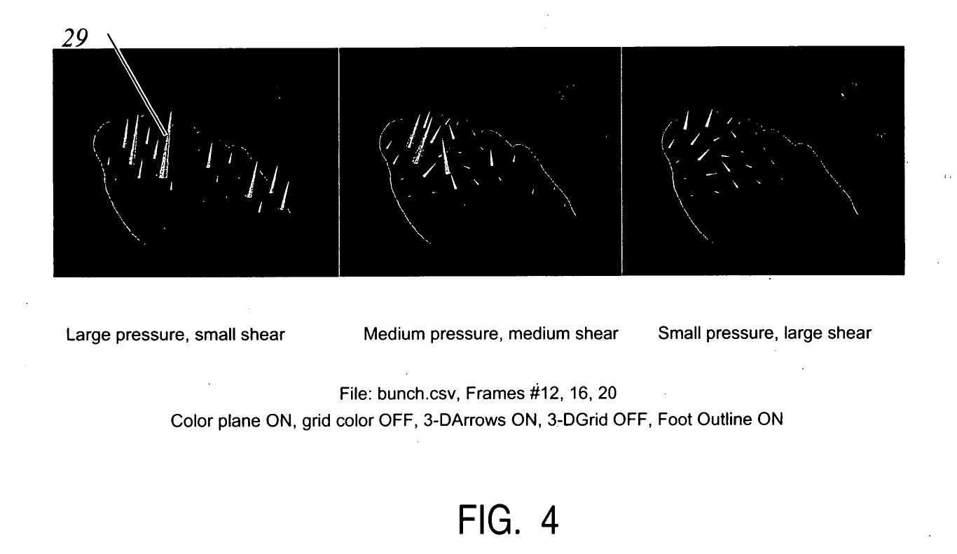

Arrows can be viewed as the sole representation of shear by turning off the grid display (see FIG. 3), but we found them most effective as an enhancement to the deformed grid, not as a substitute. The arrows used for the clinical evaluation lie in the horizontal plane. We also experimented with arrows 29 oriented in three dimensions that represent the vector direction and magnitude of the composite force vector (x, y shear and z pressure) as a way to combine vertical pressure and horizontal shear (see FIG. 4). This rendering has some interesting properties that might be worth pursuing as a secondary visual enhancement, but was not judged as an appropriate primary mechanism to visualize vertical pressure.

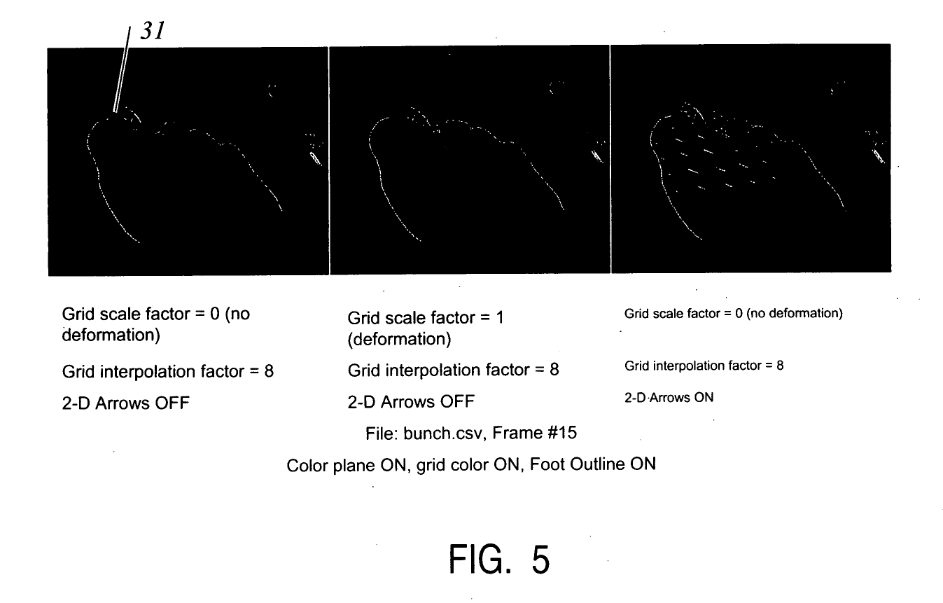

Pressure Data Visualization (simultaneous with shear). Existing pressure data visualization software uses either color mapping or a wire mesh topology map of vertical height (or both) to display vertical pressure. As anticipated, if we attempt to use a grid 31 elevated in the vertical direction to provide a topology map representing pressure at the same time we deform the grid in the horizontal plane to represent shear, the two types of grid deformation become confused (see FIG. 5). Therefore, we focus on the use of color mapping to represent vertical pressure while we used the grid deformation for horizontal shear.

The pressure data is visualized using linear color mapping, which works by defining the color of a minimum value and the color of a maximum value. Any value between the specified minimum and maximum is represented by a shade of color between the minimum color and the maximum color. Any value outside of the minimum and maximum is clamped and represented by the minimum or maximum color. We specify the start and end colors of the range using the hue, saturation, and value (HSV) system. We also allow the transparency to vary linearly through the range. This can provide a useful effect where low pressure values are mostly transparent, making them appear in the black background color. Initially, we limited ourselves to linear variations of the color parameters through the selected data range. Alternatively, we could also provide for custom look-up tables to provide nonlinear mapping of color to pressure values.

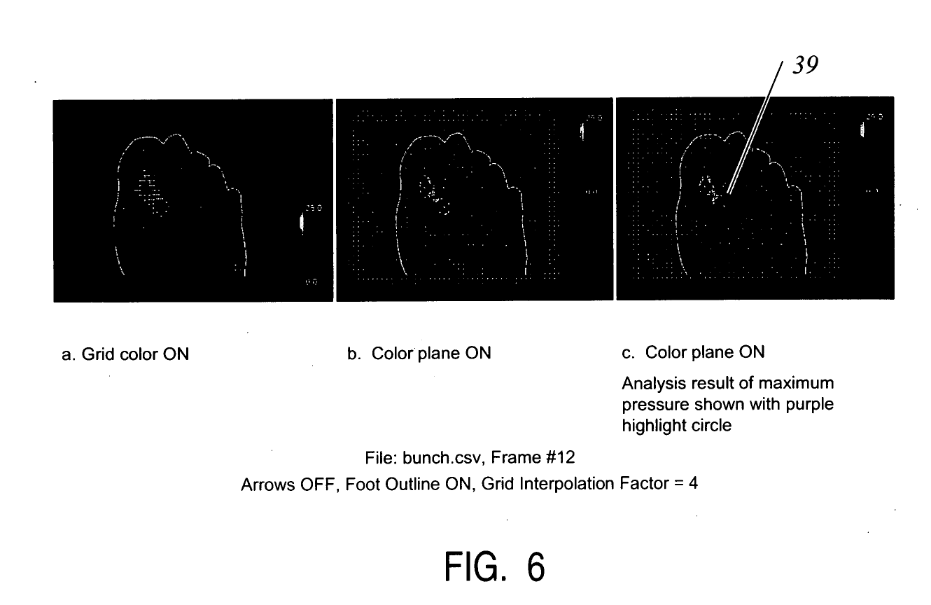

The software can apply the color map of pressure values in one of two ways: either to the grid lines themselves (grid color) or to the horizontal plane (color plane). Both approaches were implemented as user options. When grid coloring by pressure value is enabled, the grid is colored to reflect the pressure applied at each point. The user can define the linear color map used to color the grid. This method of visualizing pressure data can be deceiving if there is significant grid deformation (when the grid is deformed, the location where the grid lines intersect does not represent the location where the pressure was read). For this reason, this is not the preferred visualization approach. See FIG. 6a for an example of the grid color option. When this option is turned off, the grid appears as a uniform (light gray) color.

FIG. 6b shows the use of the color plane option. When enabled, a plane is created behind the grid that is colored to reflect the pressure applied at each point. Again, the user can define the linear color map used to color the plane. The color at the location where the undeformed grid lines intersect always represents the pressure that is measured at that point (unless it is an interpolated point, in which case, the pressure is linearly interpolated from the surrounding four points).

FIGS. 1 and 6b depict the simultaneous visualization of shear by 2-D mesh deformation and vertical pressure using a color plane.

Analysis Algorithms and Software

Since sensor data sets can be rather large, visualization alone is not always the most effective way to spot patterns of interest in the data. Consequently, one of the present invention's specific aims is to provide algorithms to automatically scan the data for certain features of interest.

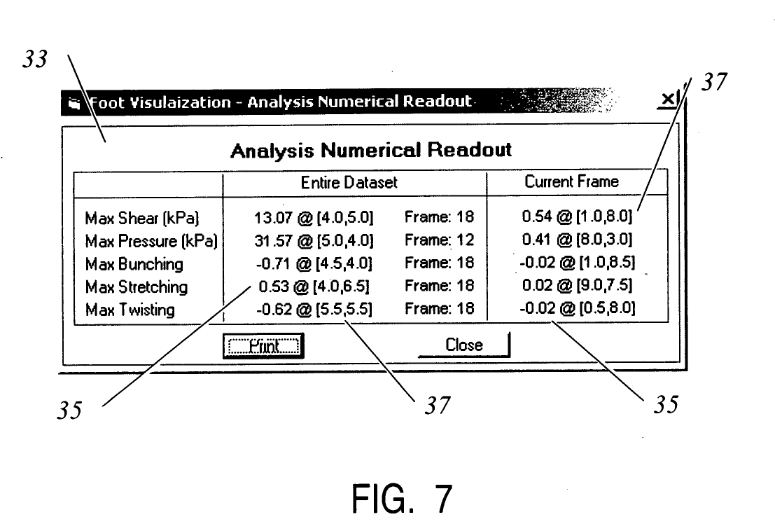

A data file that is open for visualization typically contains more than one frame, or time slice, of data. When performing analysis, we can analyze the data set (all data in the file), and, at other times, only analyze the frame currently being viewed. Results of both types of analysis are shown in a numerical readout window 33 when selected on the control panel (see FIG. 7).

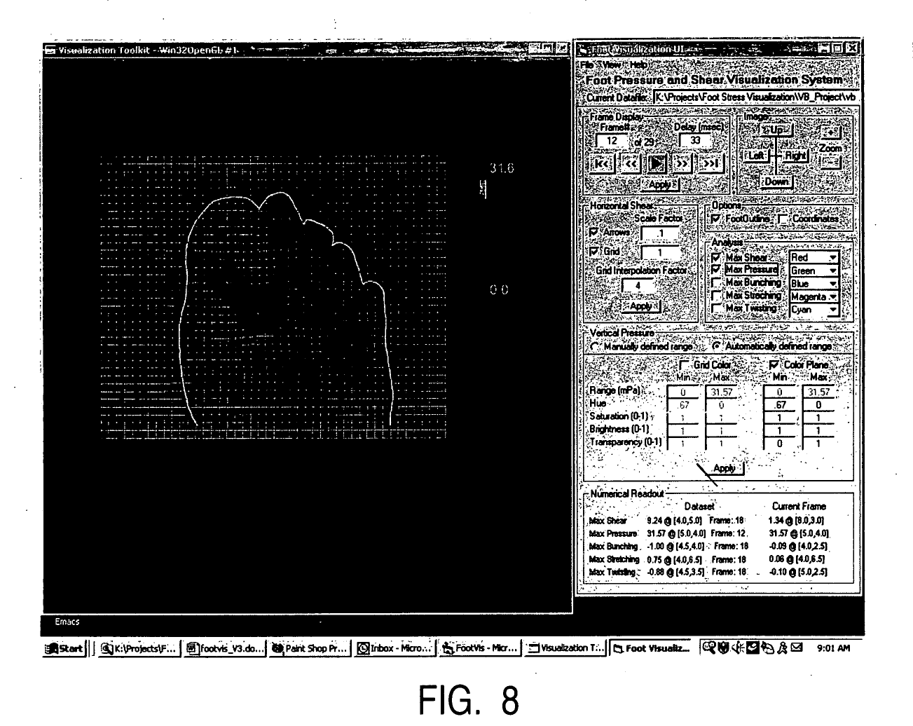

The analysis algorithms and software provide a quantitative way of measuring the maximum value 35 of shear, pressure, bunching, stretching and twisting, and provides the x-y locations 37 where these maximums occur. To better visualize the analysis results, the location of any of these maximum values (either for the current frame or the entire data set) can be highlighted in the rendering window. When selected, a colored circle 39 or other marker is placed at the x-y location of the analysis feature maximum. If highlighting a maximum for the current frame, the highlighted circle location will move as the user selects a different frame for viewing (or if the frame display is animated). If highlighting a maximum for the entire data set, the circle will always stay on the same x-y coordinate, even though that does not correspond to the maximum for the displayed frame. See FIGS. 5, 6c and 8 for examples of analysis highlighting.

Five types of analysis can be performed on the data set and the current frame, and the methods used to find each type are described below.
1. Maximum (Max) Shear: found by combining the x and y shear values using the root of the sum of the squares, and using the largest value as the max shear.
2. Max Pressure: found by comparing all pressures and using the largest pressure as the max pressure.
3. Max Bunching: the degree of bunching is measured by comparing the change in x shear (x shear of right sensor minus x shear of left sensor) and the change in y shear (y shear of top sensor minus y shear of bottom sensor). The maximum bunching is located where the largest negative change in shear (x or y) occurs.
4. Max Stretching: the degree of stretching is measured by comparing the change in x shear (x shear of right sensor minus x shear of left sensor) and the change in y shear (y shear of top sensor minus y shear of bottom sensor). The maximum bunching is located where the largest positive change in shear (x or y) occurs.
5. Max Twisting: the degree of twisting is measured by summing the four adjacent shearing values in the clockwise direction. (Subtract the y-shear of the sensor to the right, subtract the x-shear of the sensor to the bottom, add the y-shear of the sensor to the left, and add the x-shear of the sensor to the top). The maximum twisting is located where the largest amount of twisting occurs (positive twisting is clockwise, and negative twisting is counterclockwise).

Initially, some of the analysis routines were implemented with simplifications. For example, the stretching and bunching indices only look along the x- and y-axes rather than computing a more general finite difference to establish the gradient of shear. If desired, the underlying mathematics can be further refined in known fashion.

System Architecture

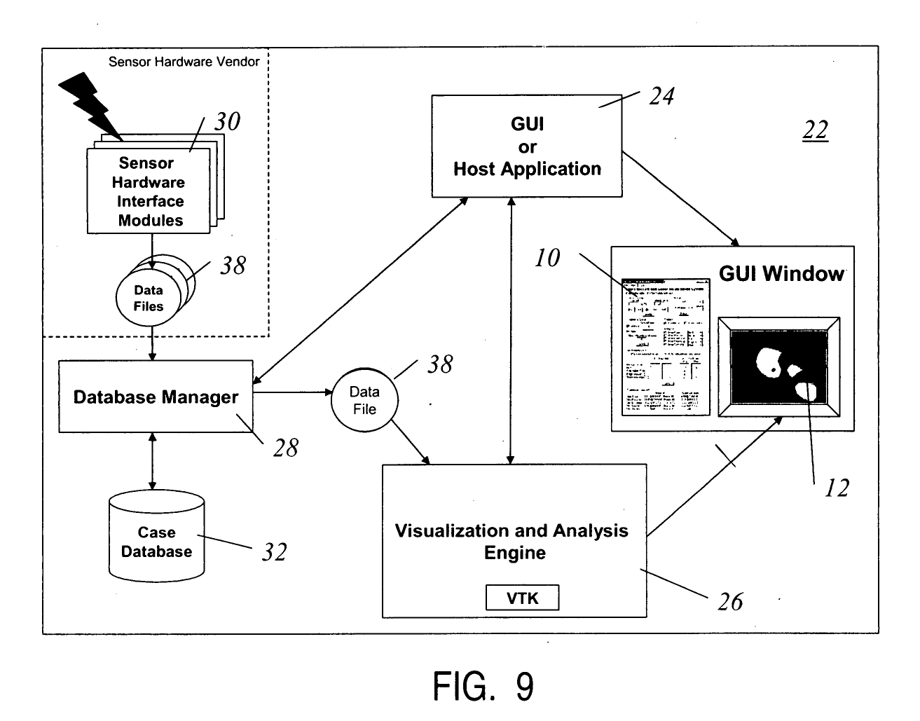

FIG. 9 illustrates the software architecture of the present invention and depicts the relationships between the individual modules. As shown, the architecture 22 consists of both a Graphical User Interface (GUI) module 24 for user control, as well as a Visualization and Analysis Engine (VAE) module 26 for data analysis and rendering capability. The architecture also contains a Database Manager module 28 to facilitate storage, categorization, and retrieval of large numbers of case files. The VAE, as well as the Database Manager, are implemented as a Windows dynamic link library (DLL). This modular approach allows the GUI and or the Database Manager to be replaced with any host application that can access the VAE DLL. Such an approach allows commercialization either as a complete turnkey application with the GUI and Database Manager, or by separate licensing of the VAE to vendors who wish to integrate it with their own GUI and database interface to preserve other existing functionality in legacy code.

The software architecture is targeted for a Windows platform, and utilizes the Visualization Toolkit (VTK) for data rendering, along with its native C++ interface. Using VTK allows us to develop rendering software using an open source, freely available, object-oriented software system, while using C++ has allowed us to gain advantages in performance, software configuration control, and deployment in a commercial environment.

The software architecture contains a set of sensor hardware interface modules 30 to acquire the foot visualization data sets; the Database Manager 28 that allows Host applications to store and retrieve the data files in a Case Database 32, the GUI or Host Application 24 that performs the display and control aspect of the VAE, as well as maintains the data files; and the VAE 26 that performs all data set rendering and analysis. The GUI or Host Application controls the main user interface window 10, while the VAE 26 controls the VTK rendering window 12 that can be embedded inside the main GUI window.

Sensor Hardware Interface Modules. The modular architecture is designed to support a variety of sensor hardware systems that may vary in quantity, size, and configuration of sensors, as well as support different data acquisition rates. All hardware-specific data conversions are handled in a sensor hardware interface module 30 that is written by, or in cooperation with, the hardware vendor. Each sensor hardware interface module can either provide direct interface to sensor hardware or import and reformatting of data collected from the hardware in an off-line mode. Any required sensor calibration is performed in the sensor hardware interface modules, with results expressed in standard engineering units for pressure and shear.

A sensor hardware interface module 30 conveys three classes of information to the remainder of the system: sensor hardware profile data, patient case data, and the actual pressure and shear data values. This information is preferably put into a data file 38 that adheres to a published open standard format. For example, the system could use the industry-standard extensible markup language (XML) as a basis for this file format. Sensor hardware profile data defines all characteristics of the model of sensor array used, such as the sensor array dimensions, resolution, and measurement ranges. This might also include data specific to the individual array used to collect the data, such as when the array was purchased or last calibrated. Patient case data includes patient identification (name, ID number, etc.) data and time of the test, test technician identification and identification of the physician requesting the test. The actual pressure and shear data values are encoded in a format that supports irregular (i.e., nonrectangular) sensor arrays. When this data is subsequently used to populate the internal data model in the VAE, it is expanded into a rectangular grid of sensor data using some standard placeholder where no sensor data exists. If foot data registration data are available from the sensor system, these are also included in the data file; otherwise, it can be derived from the data itself.

Case Database and Database Manager. The Case Database 32 is a relational database that catalogs all data files produced by the hardware interface modules according to their associated patient identification and test information. The Case Database allows users to store foot pressure and shear data files in a common repository where desired information can be readily retrieved for visualization or reporting.

The Database Manager 28 provides a consistent interface for the Host Application or GUI 24 to store and retrieve data files to and from the Case Database 32. Other functions such as those to view the database contents and extract specific data for reporting is also provided in the Database Manager. The Database Manager is provided as a DLL with standard function calls so that any Host Application can access the Case Database information without requiring any knowledge of the database schema. The retrieved data files from the Case Database are identical to the ones that were initially stored into the database. This facilitates the implementation of a system that has no Database Manager or Case Database. In this scenario, data files can come directly from the Sensor Hardware Interface Modules 30 or can be exported from another database or system and can be sent directly to the VAE 26 for processing, without the need for any intervening Database Manager and Case Database.

GUI. The GUI 24 serves as the default Host Application for the VAE. It facilitates all user control over the data being rendered. The GUI is developed in a high-level graphical programming language. Either Visual Basic (VB) or Visual C++ would work well here as the Host Application to interface with the VAE, which can support any standard Microsoft language since it is implemented as a DLL. Initially, we used VB because it allowed us to quickly prototype the system, allowing the VB host application to provide the means to control the data selection, data display, and rendering options for the VAE. The GUI's interface to the VAE will be via calls to the VAE DLL. All function calls are synchronous and allow the developer full functionality to the VTK rendering and analysis capabilities of the engine, as well as accessibility to the data information in the VAE, including patient case data as well as sensor hardware profile data.

The original GUI used an extensive control panel for all operator interaction with the VAE. This was appropriate for proof-of-concept where the primary interest was in evaluating different display and analysis options. The commercial GUI will be more oriented towards patient case processing, with many of the detailed visualization controls buried under an Options or Preferences menu. A full-featured Windows interface can be provided using menus, toolbars, and tabbed option-setting panels. The GUI can contain a full context-sensitive Help facility, as well as the ability to embed the VAE Rendering Window inside the GUI window as a child window. The GUI 24 will use the Database Manager 28 to facilitate retrieval of foot visualization data for rendering or reporting purposes. The GUI will request the retrieval of a selected data file from the Case Database 32, and the data file will be recreated and passed to the VAE for manipulation and analysis.

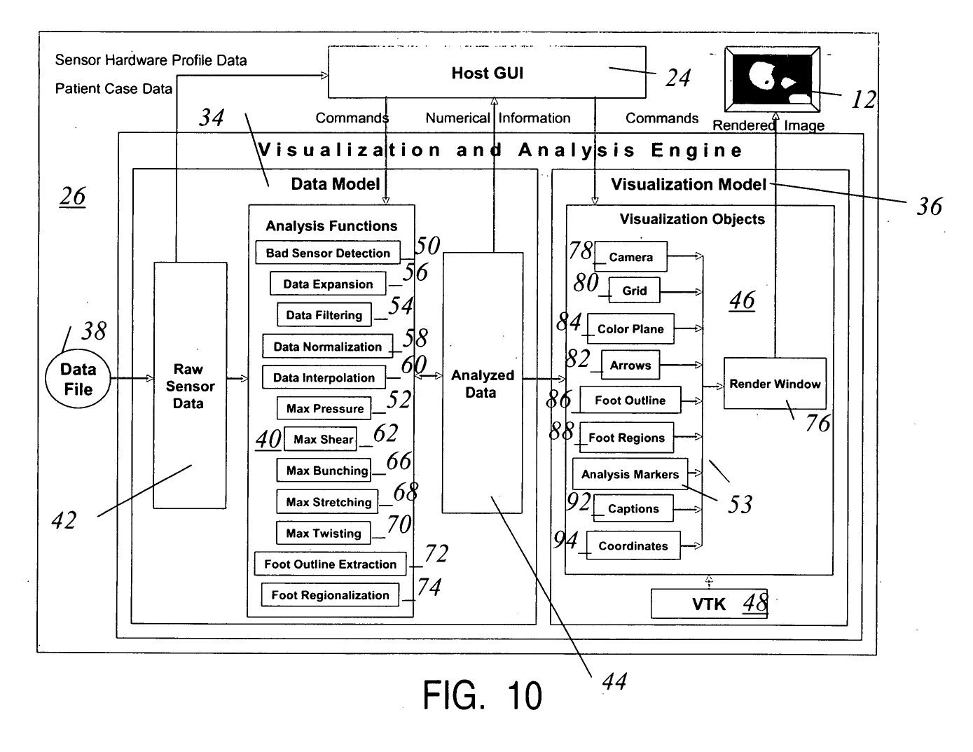

VAE. The VAE 26 serves as a tool for providing analysis and visualization of foot pressure and shear data (see FIG. 10 for an illustration of the VAE architecture). It can be implemented as a DLL written in C++, which can be easily utilized by any customer host software. The graphical rendering of the data is facilitated by VTK library calls embedded in the lower levels of the DLL and hidden from the developer. Because the engine is a DLL, the system can be packaged with our front-end GUI as a turnkey system, or the engine can be delivered alone for vendor inclusion in another host application.

The VAE 26 contains C++ functions that allow a GUI host application to pass it a data file 38 to be read and processed, and then send it commands to render the data in various ways. Visualization of pressure and shear forces, as well as identification of features in the data such as bunching, stretching, or twisting can be commanded from the Host GUI. The VAE rendering window can be viewed as a child window inside of a main GUI window or as a standalone window. Numerical information about the analyzed data, as well as sensor hardware profile data and patient case data can be sent back to the GUI for display if desired.

The VAE contains a Data Model 34 and a Visualization Model 36, which facilitate flow of data through the system from raw data file 38 entering the VAE to the rendered image 12 displayed on the GUI window. The VAE also contains analysis functions and visualization objects, which provide all analysis and visualization functionality in the engine. Upon user request, various analysis functions 40 and visualization objects 46 interact with the Data Model to provide data analysis and visualization capability. Different types of internally represented data exist at pre-analysis and post-analysis stages in the system. Raw Sensor Data 42 exists after the data file is read in, but before any analysis is performed. Analyzed Data 44 exists after the Analysis Functions 40 have manipulated the Raw Sensor Data.

VAE Data Model. The VAE includes a Data Model 34 and a Visualization Model 36 that facilitate the flow of data through the system. The Data Model consists of internally represented Raw Sensor Data 42, internally represented Analyzed Data 44, and various Analysis Functions 40 that operate on the data. The Visualization Model 36 consists of Analysis Objects 46 and the VTK 48, which together represent the objects that get visualized on the VTK Render Window 12.

Raw Sensor Data. The Raw Sensor Data module 42 consists of the vendor-supplied sensor data that enters the system via the data file 38. The data are read and stored in the Raw Sensor Data module. The module contains all three classes of information: sensor hardware profile data, patient case data, and the actual pressure and shear data values. The sensor hardware profile data and patient case data can be requested by the Host GUI at any time for display or reporting purposes. Various Analysis Functions 46 exist in the VAE that work with the Raw Sensor Data and manipulate it to produce Analyzed Data 44. An example of this might be a Bad Sensor Detection object 50 that takes the Raw Data from the Raw Sensor Data module 42, throws out any bad data associated with a bad sensor, and stores the resulting data in the Analyzed Data module 44.

Analyzed Data. The Analyzed Data module 44 consists of the analyzed data generated by each of the various Analysis Functions 40. The Analysis Functions can either start with the Raw Sensor Data and manipulate it to produce Analyzed Data as described in the previous paragraph, or they can start with previously Analyzed Data and manipulate it to produce newly Analyzed Data. An example of the second scenario might be a Max Pressure object 52 that takes the Analyzed Data from the Analyzed Data module after the data has already been expanded, filtered, and interpolated; finds the maximum pressure value over the entire time range of data; and stores that value back in the Analyzed Data module 44. In addition to storing analyzed data that the Analysis Functions produce, the Analyzed Data module provides the data required by the Visualization Objects. For example, to graphically display the position of the max pressure on each frame, the Analysis Markers object 53 uses the maximum pressure over the entire time range of data, which was stored in the Analyzed Data module 44 by the Max Pressure object 52. The Host GUI can request some of the data in the Analyzed Data module, such as the Maximum Pressure and Shear, at any time for display or reporting purposes.

Analysis Functions. All Analysis Functions 40 are invoked via commands from the Host GUI and will provide various analysis capabilities in the VAE. Bad Sensor Detection 50 and Data Filtering 54 are Analysis Functions that will increase sensor data reliability and consistency. At the user's request, all data associated with bad sensors will be optionally removed and replaced with interpolated data with appropriate indication of the bad sensor carried along in the data model. Data Filtering capabilities can optionally be applied over time or space using forward and reverse 2-D Fast Fourier Transform (FFT) processing to alter frequency content. Support for differing sensor array resolutions is provided by the Data Expansion function 56, which constructs a rectangular grid from any given sensor pattern and provides for dynamic allocation of all memory data structures based on the number of actual sensors employed. The Data Normalization 58 and Data Interpolation 60 functions are also used to increase effective resolution of the hardware and allow researchers to compare data from different hardware arrays at equivalent apparent resolutions. Max Shear 62, Pressure 52, Bunching 66, Stretching 68, and Twisting 70 functions determine where these features occur over the entire time range of data or within a frame. The Foot Outline Extraction 72 function determines the position of the foot from the analyzed data, and the Foot Regionalization function 74 partitions the analyzed foot data into predefined regions of interest in which other analysis functions can be applied.

VAE Visualization Model. The Visualization Model 36 is made up of a set of VTK Visualization Objects 46 which all represent something that can be displayed in the VTK Render Window upon user request. The Render Window Object 76 is one exception to this because this object enables all the other Visualization Objects to be displayed in the VTK Render Window 12. The VTK Render Window can be either a standalone window on the Host GUI screen, or it can be handled as a child window of one of the existing GUI windows.

Visualization Objects. The Visualization Objects 46 work from the data in the Analyzed Data module 44 to create various objects that will eventually be rendered in the VTK render window 12. All Visualization Objects are an instantiation of VTK objects or a combination of VTK objects. Upon command from the GUI, these objects are sent to the Render Window Object 76 to be displayed in the VTK Render Window 12.

These objects will provide various visualization capabilities in the VAE. The Camera object 78 provides rotation, translation, and zoom capabilities in the render window. The Grid 80 and Arrows 82 objects display the Horizontal Shear in the render window, while the Color Plane object 84 facilitates the visualization of vertical pressure in color. The Foot Outline 86 and Foot Region 88 objects encapsulate the visualization of the outline of the foot as well as the regions of the foot. The Analysis Markers object 53 displays a colored circular marker at locations where user-specified analysis features occur. The Caption object 92 consists of text strings that contain various types of information (i.e., sensor hardware profile data, patient case data), which can be positioned by the user on the VTK render window 12. The Coordinates object 94 allows visualization of the grid axis labels.

The above described system displays shear and pressure data in a combined visualization, and analyzes interactions of shear and pressure on the plantar surface to identify and characterize skin stress patterns (such as bunching, shearing, twisting or stretching) believed to create a high risk of ulceration. This new visualization system can interface with a variety of pressure/shear platform hardware and facilitates data storage, analysis and display. Accordingly, the present invention provides a key tool for research in the etiology of foot ulcers and clinical evaluation of risk factors in diabetic patients. This system is also applicable to the study, visualization and evaluation of other skin/surface interactions including bed sores, saddle sores, skin breakdown when wearing a prosthetic limb in patients with known peripheral vascular disease, etc.

Although a particular, presently preferred embodiment has been described and depicted, those skilled in the art will recognize that various modifications, additions and substitutions can be made without departing from the spirit of the invention.

1. A method for visualizing force acting on a skin surface, the force being sensed at distributed points on the skin surface by an array of sensors, comprising:
displaying an image of a mesh; and
deforming the mesh in a plane of the mesh in accordance with the force sensed by the array of sensors.

2. The method of claim 1, further comprising:
superimposing the image of the mesh on an outline of the skin surface.

3. The method of claim 1, wherein the mesh comprises a rectangular grid and intersections of lines of the grid when undeformed correspond to locations of said sensors in said array.

4. The method of claim 3, wherein points on outside edges of the grid comprise anchor points which create a fixed reference that frames the image.

5. The method of claim 4, wherein the anchor points are represented with zero force.

6. The method of claim 3, wherein the intersections of the lines of the grid in the image are displaced in proportion to the force measured at said sensors.

7. The method of claim 1, wherein the force comprises shear.

8. The method of claim 7, wherein the skin surface comprises a plantar surface of a foot.

9. The method of claim 1, further comprising:
superimposing on the image of the mesh, a field of scaled arrows, wherein an arrow points in a direction of a force vector and a relative magnitude of an arrow is proportional to a sensed force magnitude.

10. The method of claim 1, further comprising:
sensing a second force acting on distributed points on said skin surface; and
using color mapping to display the sensed second force along with said image.

11. The method of claim 10, wherein the second force comprises force or pressure acting generally normal to said surface, and said color mapping comprises color mapping of at least one of the mesh and a plane parallel to said mesh.

12. The method of claim 1 1, wherein said color mapping comprises linear color mapping.

13. The method of claim 1, further comprising:
automatically determining a location of maximum value of at least one of:
said measured force, skin bunching, skin stretching and skin twisting; and
highlighting said location in said image.

14. The method of claim 10, further comprising:
automatically determining a location of maximum value of at least one of:
said measured forces, skin bunching, skin stretching and skin twisting; and
highlighting said location on said image.

15. The method of claim 14, wherein the measured force comprises shear, and the measured second force comprises pressure.

16. The method of claim 10, wherein said force and said second force are measured with the same array of sensors.

17. The method of claim 16, wherein said force comprises shear, and said second force comprises pressure.

18. A method for visualizing forces acting on a skin surface, as detected at distributed points on said skin surface with an array of sensors, comprising:
automatically providing a first representation of detected forces acting in a plane of said skin surface; and
simultaneously automatically providing a second representation of detected forces acting in a direction generally normal to said skin surface.

19. The method of claim 18, wherein said first representation portrays shear and said second representation portrays pressure.

20. The method of claim 19, wherein said first representation comprises a mesh deformed within a plane of the mesh, and said second representation comprises color mapping.

21. The method of claim 18, wherein said first representation comprises directed scaled arrows.

22. The method of claim 18, wherein said first representation and said second representation comprise common arrows having a vector direction and magnitude corresponding to a composite vector of all detected forces.

23. The method of claim 18, further comprising:
automatically determining a location of maximum value of at least one of: the detected forces, skin bunching, skin stretching, and skin twisting; and
highlighting said location.

24. The method of claim 18, wherein said skin surface comprises a plantar surface of a foot, and further comprising providing a representation of said plantar surface along with said first and second representations.

25. A system for visualizing forces acting on a skin surface, comprising:
a visualization and analysis engine for receiving data files containing force readings from an array of sensors at distributed locations of said skin surface, analyzing said data files and generating a first representation of forces acting in a plane of said skin surface and a second representation of forces acting generally normal to said skin surface; and
a rendering window for simultaneously displaying said first and second representations.

26. The system of claim 25 further comprising:
a graphical user interface for user control of the visualization and analysis engine;
data base manager for managing storage, categorization and retrieval of data files; and
sensor hardware interface modules for acquiring data files from a variety of different sensor hardware systems.

27. The system of claim 25, wherein said visualization and analysis engine comprises a data model and a visualization model; the data model comprising raw sensor data, analysis functions, and analyzed data; and the visualization model comprises visualization objects which operate on the analyzed data to create said first and second representations in said rendering window.

28. The system of claim 27, wherein said visualization objects include a mesh portrayed in said first representation, and a color plane for said second representation.

29. An article of manufacture comprising a computer useable medium having computer readable program code means embodied therein for a system for visualizing forces acting on a skin surface, said article of manufacture comprising:
computer readable program code means for establishing a visualization and analysis engine for receiving data files containing force readings from an array of sensors at distributed locations of said skin surface, analyzing said data files and generating a first representation of forces acting in a plane of said skin surface and second representation of forces acting normal to said skin surface; and
computer readable program code means for causing said visualization and analysis engine to simultaneously display said first and second representations in a rendering window.

30. The article of manufacture of claim 29 wherein said visualization and analysis engine comprises a data model and a visualization model; the data model comprises analysis functions for operating on raw sensor data to produce analyzed data; and the visualization model comprises visualization objects which operate on the analyzed data to create said first and second representations in said rendering window.

31. The article of manufacture of claim 30 wherein said visualization objects include a mesh portrayed in said first representation, and a color plane for said second representation.

32. The method of claim 1, further comprising: providing an array of sensors for sensing the force at the distributed points on the skin surface.

33. The method of claim 18, further comprising: providing an array of sensors for detecting the force at the distributed points on the skin surface.

34. The system of claim 25, in combination with an array of sensors for providing said force readings.
